(19) United States
(12) Patent Application Publication
(10) Pub. No.: US 2026/0305671 A1
(43) Pub. Date: Oct. 8, 2026

(54) SOLID FORMULATIONS COMPRISING BETA-CAROTENE

(71) Applicant: DSM IP ASSETS B.V., Maastricht (NL)

(72) Inventors: Antonio ESTRELLA, Kaiseraugst (CH); Markus NOWOTNY, Kaiseraugst (CH); Christian SCHAEFER, Kaiseraugst (CH); Roland SCHEX, Kaiseraugst (CH); Bernd SCHLEGEL, Kaiseraugst (CH)

(21) Appl. No.: 19/100,718

(22) PCT Filed: Aug. 2, 2023
(86) PCT No.: PCT/EP2023/071431

§ 371 Date: Feb. 3, 2025

(30) Foreign Application Priority Data
Aug. 5, 2022 (EP) 22020377.2

(51) Int. Cl. A23L 33/155; A23L 29/219

(52) U.S. Cl. CPC A23L 33/155; A23L 29/219

(57) ABSTRACT

The present invention relates to a solid formulation comprising β-carotene with a high amount of a specific stereochemical form. Furthermore, the present invention relates to a process of production of such a specific formulation.

The present invention relates to a solid formulation comprising β-carotene wherein the β-carotene is present in a high amount of a specific stereochemical form (all E form). Furthermore, the present invention relates to a process of production of such a specific formulation.

β-Carotene, which is the compound of formula (I)

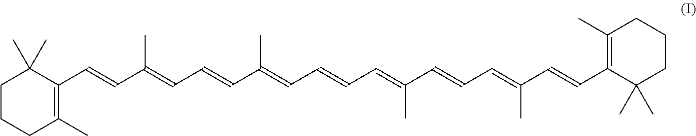 [SMILES: CC1=C(/C=C/C(C)=C/C=C/C(C)=C/C=C/C=C(C)/C=C/C=C(C)/C=C/C2=C(C)CCCC2(C)C)C(C)(C)CCC1]

is an important and useful compound. The compound for formula (I) is as shown in the “all-E” stereochemical form.

Due to the carbon-carbon double bonds, which are part of the structure of β-carotene, there are a variety of stereochemical forms of β-carotene.

Very desired one is the all-E form (as shown above). This stereochemical form of β-carotene is known to be a good bioavailable form.

Furthermore, the all-E form of β-carotene shows a better provitamin A activity than other stereochemical forms.

In the body, β-carotene converts into vitamin A (retinol). Vitamin A is important for good vision and eye health, for a strong immune system, and for healthy skin and mucous membranes.

Next to the health benefits, β-carotene can also be used as a colorant, especially to color food and beverages.

β-Carotene as such can be sourced from natural sources or it can be produced chemically or biochemically.

Due to the fact that β-carotene can be used for a variety of applications, β-carotene needs to be formulated. This means that β-carotene is put in a formulation, which is suitable for that specific application.

β-Carotene can be formulated as liquid emulsions as well as solid formulations, such as spray-dried powders, granules, beadlets or similar dried product formulations. These formulations can then be used as such, or they can be further formulated into the final product form.

The present invention relates to specific solid formulations.

The solid formulations of the present invention can be implemented in a food product, a beverage, an animal feed product, a consumer product, or a health product.

To produce solid formulations, usually in a first step an emulsion/dispersion comprising β-carotene is produced, which is then in a second step transformed into a solid formulation using commonly known technologies, such as drying, spray drying, etc.

A problem, which occurs when producing formulations comprising β-carotene, is that β-carotene can easily isomerize into the various stereochemical forms.

The goal of the present invention was to provide a solid formulation comprising β-carotene, wherein the content of β-carotene having the important all-E form is at least 55% (based on the total content of the β-carotene).

Therefore, the present invention relates to a solid formulation (SF) comprising
(i) β-carotene, and
(ii) at least one hydrocolloid, and
(iii) tocopherol, and
(iv) optionally water
(v) optionally at least one additional ingredient,
(vi) optionally a coating layer,
   characterised in that at least 55 weight-% (wt-%) of the β-carotene (based on the total weight of the β-carotene) is in the all-E form.

The β-carotene used in the formulation according to the present invention can be from any source. This means that β-carotene can be from a natural source as well as be produced via chemical synthesis or biotechnological processes.

It is also possible to use a mixture of natural and nature-identical β-carotene from the above-mentioned production processes.

For the present invention the source of the β-carotene is not critical.

Essential for the present invention is that the content of the β-carotene in the all-E form is at least 55 wt-%, based on the total weight of the β-carotene.

Preferably, the content of the β-carotene in the all-E form is at least 58 wt-%, based on the total weight of the β-carotene; more preferably the content of the β-carotene in the all-E form is at least 60 wt-%, based on the total weight of the β-carotene, especially preferred the of the content of β-carotene in the all-E form is at least 62 wt-%, based on the total weight of the β-carotene.

Preferably, the solid formulation (SF) has been obtained by using spray-granulation, preferably by using a spray-granulation step starting from a liquid emulsion comprising at least the ingredients of said solid formulation (i) through (iii), and optionally ingredients (iv) and (v).

Therefore, the present invention relates to a solid formulation (SF1), which is formulation (SF), wherein the content of the β-carotene in the all-E form is at least 58 wt-%, based on the total weight of the β-carotene.

Therefore, the present invention relates to a solid formulation (SF1′), which is formulation (SF), wherein the content of the β-carotene in the all-E form is at least 60 wt-%, based on the total weight of the β-carotene.

Therefore, the present invention relates to a solid formulation (SF1″), which is formulation (SF), wherein the content of the β-carotene in the all-E form is at least 62 wt-%, based on the total weight of the β-carotene.

The overall content of the β-carotene in the solid formulation according to the present invention is 0.5 to 25 wt-%, based on the total weight of the solid formulation.

Preferably, the overall content of the β-carotene in the solid formulation according to the present invention is 1 to 25 wt-%, based on the total weight of the solid formulation.

More preferably, the overall content of the β-carotene in the solid formulation according to the present invention is 1 to 20 wt-%, based on the total weight of the solid formulation.

Especially preferred, the overall content of the β-carotene in the solid formulation according to the present invention is 1 to 15 wt-%, based on the total weight of the solid formulation.

Most preferred, the overall content of the β-carotene in the solid formulation according to the present invention is 2 to 15 wt-%, based on the total weight of the solid formulation.

Therefore, the present invention relates to a solid formulation (SF2), which is formulation (SF), (SF1), (SF1′) or (SF1″), wherein the content of the β-carotene in the solid formulation is 0.5 to 25 wt-%, based on the total weight of the solid formulation.

Therefore, the present invention relates to a solid formulation (SF2′), which is formulation (SF), (SF1), (SF1′) or (SF1″), wherein the content of the β-carotene in the solid formulation is 1 to 25 wt-%, based on the total weight of the solid formulation.

Therefore, the present invention relates to a solid formulation (SF2″), which is formulation (SF), (SF1), (SF1′) or (SF1″), wherein the content of the β-carotene in the solid formulation is 1 to 20 wt-%, based on the total weight of the solid formulation.

Therefore, the present invention relates to a solid formulation (SF2″), which is formulation (SF), (SF1), (SF1′) or (SF1″), wherein the content of the β-carotene in the solid formulation is 1 to 15 wt-%, based on the total weight of the solid formulation.

Therefore, the present invention relates to a solid formulation (SF2″″), which is formulation (SF), (SF1), (SF1′), (SF1″) or (SF1′″), wherein the content of the β-carotene in the solid formulation is 2 to 15 wt-%, based on the total weight of the solid formulation.

The solid formulation according to the present invention comprises at least one hydrocolloid. The hydrocolloids according to the present invention have emulsifying properties.

In the context of the present invention, hydrocolloids are polysaccharides, gelatin of low bloom, medium bloom or high bloom from fish, pork, or bovine, caseins/caseinates, plant- or animal-based protein, other proteinaceous hydrocolloids, and lignosulfonate.

Preferred hydrocolloids according to the invention are polysaccharides.

In the context of the present invention, the term polysaccharide as used herein includes xanthan gum, gum acacia, pectin, guar gum, caroub gum, alginates, celluloses, cellulose derivatives, such as starch and starch derivatives. Preferred polysaccharides according to the present invention are gum acacia, starch, starch derivatives, more preferred are gelatinized starch and modified food starch and especially preferred are modified food starches.

In the context of the present invention, the term “modified food starch” as used herein relates to modified starches that are made from starches substituted by known chemical methods with hydrophobic moieties. For example, starch may be treated with cyclic dicarboxylic acid anhydrides such as succinic and/or glutaric anhydrides, substituted with an alkyl or alkenyl hydrocarbon group.

A very common and preferred modified starch is starch sodium octenyl succinate (OSA-starch). OSA-starch as used herein denotes any starch (from any natural source such as corn, wheat, tapioca, potato or synthesized) that was treated with octenyl succinic anhydride. The degree of substitution, i.e., the number of esterified hydroxyl groups with regard to the total number of hydroxyl groups usually varies in a range of from 0.1 percent to 10 percent, preferably in a range of from 0.5 percent to 5 percent, more preferably in a range of from 2 percent to 4 percent.

OSA-starches are commercially available e.g., from Ingredion under the trade names HiCap 100, Capsul HF, Capsul HS, Purity Gum 2000, UNI-PURE, HYLON VII from Roquette Freres; from Cargill under the trade name C*EmCap or from Tate and Lyle.

The content of the at least one hydrocolloid is usually from is from 40 to 90 wt-%, based on the total weight of the solid formulation, preferably from 45 to 90 wt-%, more preferably from 50 to 90 wt-%, based on the total weight of the solid formulation.

Therefore, the present invention relates to a solid formulation (SF3), which is the solid formulation (SF), (SF1), (SF1′), (SF1″), (SF2), (SF2′), (SF2″), (SF2″) or (SF2″″), wherein the at least one hydrocolloid is chosen from the group consisting of polysaccharides, gelatin of low bloom, medium bloom or high bloom from fish, pork or bovine, caseins/caseinates, plant- or animal-based protein, other proteinaceous hydrocolloids, and lignosulfonate.

Therefore, the present invention relates to a solid formulation (SF3′), which is the solid formulation (SF), (SF1), (SF1′), (SF1″), (SF2), (SF2′), (SF2″), (SF2′″) or (SF2″″), wherein the at least one hydrocolloid is chosen from the group consisting of xanthan gum, gum acacia, pectin, guar gum, caroub gum, alginates, celluloses, cellulose derivatives, such as starch and starch derivatives.

Therefore, the present invention relates to a solid formulation (SF3″), which is the solid formulation (SF), (SF1), (SF1′), (SF1″), (SF2), (SF2′), (SF2″), (SF2″) or (SF2″″), wherein the hydrocolloid is modified food starch.

Therefore, the present invention relates to a solid formulation (SF3″′), which is the solid formulation (SF3″), wherein the modified food starch is starch sodium octenyl succinate.

Therefore, the present invention relates to a solid formulation (SF4), which is the solid formulation (SF), (SF1), (SF1′), (SF1″), (SF2), (SF2′), (SF2″), (SF2″′), (SF2″″), (SF3), (SF3′), (SF3″) or (SF3″′), wherein the content of the at least one hydrocolloid is 40 to 90 wt-%, based on the total weight of the solid formulation.

Therefore, the present invention relates to a solid formulation (SF4′), which is the solid formulation (SF), (SF1), (SF1′), (SF1″), (SF2), (SF2′), (SF2″), (SF2″′), (SF2″″), (SF3), (SF3′), (SF3″) or (SF3″′), wherein the content of the at least one hydrocolloid is 45 to 90 wt-%, based on the total weight of the solid formulation.

Therefore, the present invention relates to a solid formulation (SF4″), which is the solid formulation (SF), (SF1), (SF1′), (SF1″), (SF2), (SF2′), (SF2″), (SF2″′), (SF2″″), (SF3), (SF3′), (SF3″) or (SF3″′), wherein the content of the at least one hydrocolloid is 50 to 90 wt-%, based on the total weight of the solid formulation.

The solid formulation according to the present invention comprises tocopherol. In the context of the present invention the term “tocopherol” encompasses any of its 8 stereoisomers, i.e., all-rac-α-tocopherol, including D/L-α-tocopherol and mixed tocopherol.

The content of the tocopherol is from 0.1 to 5 wt-%, based on the total weight of the solid formulation, preferably from 0.1 to 4 wt-%, more preferably from 0.5 to 3 wt-%, based on the total weight of the solid formulation.

Therefore, the present invention relates to a solid formulation (SF5), which is the solid formulation (SF), (SF1), (SF1′), (SF1″), (SF2), (SF2′), (SF2″), (SF2′″), (SF2″″), (SF3), (SF3′), (SF3″), (SF3″′), (SF4), (SF4′) or (SF4′), wherein the content of the tocopherol is 0.1 to 5 wt-%, based on the total weight of the solid formulation.

Therefore, the present invention relates to a solid formulation (SF5′), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′) or (SF4′), wherein the content of the tocopherol is 0.1 to 4 wt-%, based on the total weight of the solid formulation.

Therefore, the present invention relates to a solid formulation (SF5″), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′) or (SF4′), wherein the content of the tocopherol is 0.5 to 3 wt-%, based on the total weight of the solid formulation.

The solid formulation according to the present invention can comprise some water (residual water). The content of the residual water can vary depending on the kind and properties of drying, which is used to dry the solid formulation.

The water content of the solid formulation according to the present invention is less than 8 wt-%, based on the total weight of the solid formulation.

Preferably, the water content of the solid formulation according to the present invention is less than 7 wt-%, more preferably less than 6 wt-%, especially preferred less than 5 wt-%, based on the total weight of the solid formulation.

Therefore, the present invention relates to a solid formulation (SF6), which is the solid formulation (SF), (SF1), (SF1′), (SF1″), (SF2), (SF2′), (SF2″), (SF2′″), (SF2″″), (SF3), (SF3′), (SF3″), (SF3″′), (SF4), (SF4′), (SF4′), (SF5), (SF5′) or (SF5″), wherein the content of water is less than 8 wt-%, based on the total weight of the solid formulation.

Therefore, the present invention relates to a solid formulation (SF6′), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′) or (SF5″), wherein the content of water is less than 7 wt-%, based on the total weight of the solid formulation.

Therefore, the present invention relates to a solid formulation (SF6″), which is the solid formulation (SF), (SF1), (SF1′), (SF1″), (SF2), (SF2′), (SF2″), (SF2″′), (SF2″″), (SF3), (SF3′), (SF3″), (SF3′″), (SF4), (SF4′), (SF4′), (SF5), (SF5′) or (SF5″), wherein the content of water is less than 6 wt-%, based on the total weight of the solid formulation.

Therefore, the present invention relates to a solid formulation (SF6′″), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′) or (SF5″), wherein the content of water is less than 5 wt-%, based on the total weight of the solid formulation.

The solid formulation according to the present invention can comprise optionally at least one further ingredient. Such ingredients can be starch hydrolysates (such as dextrins, maltodextrins and glucose syrup), oils (such as coconut oil, sunflower oil, corn oil, MCT oil and other vegetable oils), dyestuffs, fillers, binders, flavours, etc.

The content of these additional ingredients can be up to 15 wt-%, based on the total weight of the solid formulation.

Therefore, the present invention relates to a solid formulation (SF7), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″) or (SF6″′), wherein the solid formulation comprises at least one additional ingredient chosen from the group consisting of starch hydrolysates (such as dextrins, maltodextrins and glucose syrup), oils (such as coconut oil, sunflower oil, corn oil, MCT oil and other vegetable oils), dyestuffs, fillers, binders and flavours.

Therefore, the present invention relates to a solid formulation (SF8), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′) or (SF7), wherein the content of the at least one additional ingredient is up to 15 wt-%, based on the total weight of the solid formulation.

The solid formulation according to the present invention can be coated, which means it can have a coating layer.

The coating layer can be any commonly known coating layer. It could also be a powder coating layer. Such a powder coating can be made from starch (such as corn starch).

The coating layer can be up to 45 wt-% of the solid formulation, based on the total weight of the solid formulation.

Therefore, the present invention relates to a solid formulation (SF9), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7) or (SF8), wherein solid formulation is coated with a coating layer.

Therefore, the present invention relates to a solid formulation (SF9′), which is the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7) or (SF8), wherein solid formulation is coated with a powder coating layer.

Therefore, the present invention relates to a solid formulation (SF10), which is the solid formulation (SF), (SF1), (SF1′), (SF1″), (SF2), (SF2′), (SF2″), (SF2″′), (SF2″″), (SF3), (SF3′), (SF3″), (SF3′″), (SF4), (SF4′), (SF4′), (SF5), (SF5′), (SF5″), (SF6), (SF6′), (SF6″), (SF6″′), (SF7), (SF8), (SF9) or (SF9′), wherein the coating is up to 45 wt-% of the solid formulation, based on the total weight of the solid formulation.

It is clear, that for one embodiment of the present invention the amount always adds up to 100%.

The solid formulation according to the present invention can be powderous, in the form of a granule, or in the form of a beadlet.

The particle size of the solid formulation can be form 50 μm to 1000 μm. The particle size distribution of the powderous formulation according to the mean Sauter diameter D (3; 2) is measured by a laser diffraction instrument Mastersizer 3000 (Malvern Instruments, Malvern, United Kingdom), applying the Fraunhofer model.

The size can vary. It can depend on a variety of factors, such as the production process, the intended use, etc.

The solid formulation (SF), (SF1), (SF1′), (SF1″), (SF2), (SF2′), (SF2″), (SF2′″), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7), (SF8), (SF9), (SF9′) or (SF10) according to the present invention can be used as such or further formulation into a final product.

The solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7), (SF8), (SF9), (SF9′) or (SF10) can be used in food products, in feed products, in dietary supplements, in pharmaceutical products and/or in personal care products.

Furthermore, the present invention relates to food products, feed products, dietary supplements, pharmaceutical products and personal care products comprising at least one solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7), (SF8), (SF9), (SF9′) or (SF10).

The solid formulation according to the present invention are produced by commonly known methods.

Generally, the solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7), (SF8), (SF9), (SF9′) or (SF10) according to the present invention is produced as follows:
Step (i): emulsifying/dispersing/dissolving of the β-carotene, and
Step (ii): producing a solid form out of the emulsion/dispersion of step (i).

Therefore, the present invention relates to a process (P) for a producing solid formulation (SF), (SF1), (SF1′), (SF1″), (SF2), (SF2′), (SF2″), (SF2″′), (SF2″″), (SF3), (SF3′), (SF3″), (SF3″′), (SF4), (SF4′), (SF4′), (SF5), (SF5′), (SF5″), (SF6), (SF6′), (SF6″), (SF6″′), (SF7), (SF8), (SF9), (SF9′) or (SF10), comprising
step (i): emulsifying/dispersing/dissolving of the β-carotene, and
step (ii): producing a solid form out of the emulsion/dispersion of step (i).

In the following the steps are discussed in more details.

### Step (i)

The β-carotene is dissolved in a solvent and/or a mixture of solvent.

Suitable solvents are organic solvents which have preferably a boiling point of less than 150° C., more preferably less than 140° C., especially less than 120° C. at 1013.25 hPa.

The preferred solvent is isobutyl acetate.

In the context of the present invention, a “dispersion” may be an emulsion, i.e. the particle's core may be liquid. Alternatively, the dispersion may be a suspension, i.e. the particle's core may be solid. In a typical embodiment of the present invention, however, the particle's core of the dispersion comprises both, liquid and solid compounds.

The “particles” of dispersion are too small to be seen with the naked eye. In a preferred embodiment of the invention, the particles have an average size in the range from 50 to 1000 nm, more preferably from 100 to 800 nm and more preferably from 100 to 500 nm [Average diameter of particle sizes, measured by Dynamic Light Scattering (Beckman Coulter, Delsa Nano S)]. The particles are water-dispersible despite of having a lipophilic core. This is achieved by surrounding the core with the at least one hydrocolloid having emulsifying properties (ingredient (ii)). Said surrounding is referred to as the shell of the particle.

An aqueous solution is prepared as well, which comprises water and at least one hydrocolloid (ingredient (ii)).

It is clear, that all preferences for the hydrocolloid as stated above apply here as well.

Preferably, the at least one hydrocolloid is chosen from the group consisting of xanthan gum, gum acacia, pectin, guar gum, caroub gum, alginates, celluloses, cellulose derivatives, such as starch and starch derivatives.

More preferably, the hydrocolloid is modified food starch.

Most preferably, the hydrocolloid is starch sodium octenyl succinate.

Finally, these two phases are combined usually under vigorous stirring and/or high-pressure homogenization to obtain the dispersion/emulsion.

It is clear, that the β-carotene phase as well as the water phase could also comprise further (non-essential) ingredients, such as starch hydrolysates (such as dextrins, maltodextrins and glucose syrup), oils (such as coconut oil, sunflower oil, corn oil, MCT oil and other vegetable oils), dyestuffs, fillers, binders, and flavours.

Depending on step (ii), the viscosity of the dispersion/emulsion needs to be in such a range that the granulation step can take place.

Therefore, the present invention relates to a process (P1), which is process (P), wherein the β-carotene is dissolved in at least one solvent which have a boiling point of less than 150° C.

Therefore, the present invention relates to a process (P1′), which is process (P), wherein the β-carotene is dissolved in at least one solvent which have a boiling point of less than 140° C.

Therefore, the present invention relates to a process (P1″), which is process (P), wherein the β-carotene is dissolved in at least one solvent which have a boiling point of less than 120° C.

Therefore, the present invention relates to a process (P1″′), which is process (P), wherein the β-carotene is dissolved in isobutyl acetate.

Therefore, the present invention relates to a process (P2), which is process (P), (P1), (P1′), (P1″) or (P1″′), wherein an aqueous solution is prepared, which comprises water and at least one hydrocolloid.

Therefore, the present invention relates to a process (P2′), which is process (P), (P1), (P1′), (P1″) or (P1″′), wherein an aqueous solution is prepared, which comprises water and at least one hydrocolloid, wherein the at least one hydrocolloid is chosen from the group consisting of xanthan gum, gum acacia, pectin, guar gum, caroub gum, alginates, celluloses, cellulose derivatives, such as starch and starch derivatives.

Therefore, the present invention relates to a process (P2″), which is process (P), (P1), (P1′), (P1″) or (P1″′), wherein an aqueous solution is prepared, which comprises water and at least one hydrocolloid, wherein the at least one hydrocolloid is a modified food starch.

Therefore, the present invention relates to a process (P2″), which is process (P), (P1), (P1′), (P1″) or (P1′″), wherein an aqueous solution is prepared, which comprises water and at least one hydrocolloid, wherein the at least one hydrocolloid is starch sodium octenyl succinate.

### Step (ii)

In step (ii) a solid form out of the emulsion/dispersion of step (i) is formed. This is done by a granulation process, more preferably in a spray-granulation process.

Granulation is a process in which powder particles are made by building up larger particles starting from small particles by layering these small particles with the solids-bearing liquid.

The granulation process via the spraying of emulsion onto the seed particles (made from spray drying of the emulsion) was performed for at least 30 minutes (up to several hours) and subsequent drying phase of at least 30 minutes (up to 2 h) at a product temperature of 70° C.-100° C., preferred 75° C. to 90° C. (most preferred around 80° C.).

The liquid emulsion is spray-granulated in two-steps:
i) the preparation of seed material applying spray drying with an air inlet temperature of 160° C. to 190° C. and an air outlet temperature of 70° C.-100° C. (preferred 75° C. to 90° C., most preferred around 80° C.) and
ii) the preparation of granules using a fluid bed processor (such as for example a fluid bed processor type WFP-Mini (DMR Prozess-Technologie GmbH)).

Therefore, the present invention relates to a process (P3), which is process (P), (P1), (P1′), (P1″), (P2), (P2′), (P2″) or (P2′″), wherein the liquid emulsion is spray-granulated in two-steps:
i) the preparation of seed material applying spray drying with an air inlet temperature of 160 to 190° C. and an air outlet temperature of 70° C.-100° C. and
ii) the preparation of granules using a fluid bed processor.

Therefore, the present invention relates to a process (P3′), which is process (P), (P1), (P1′), (P1″), (P2), (P2′), (P2″) or (P2″′), wherein the liquid emulsion is spray-granulated in two-steps:
i) the preparation of seed material applying spray drying with an air inlet temperature of 160 to 190° C. and an air outlet temperature of 75° C. to 90° C., and
ii) the preparation of granules using a fluid bed processor.

Therefore, the present invention relates to a process (P3″), which is process (P), (P1), (P1′), (P1″), (P2), (P2′), (P2″) or (P2″′), wherein the liquid emulsion is spray-granulated in two-steps:
i) the preparation of seed material applying spray drying with an air inlet temperature of 160 to 190° C. and an air outlet temperature of around 80° C. and
ii) the preparation of granules using a fluid bed processor.

Therefore, the present invention relates to a process (P4), which is process (P), (P1), (P1′), (P1″), (P2), (P2′), (P2″), (P2′″), (P3), (P3′) or (P3″), wherein the granulation process is performed for at least 30 minutes (up to several hours) and subsequent drying phase of at least 30 minutes (up to 2 h) at a product temperature of 70° C.-100° C. is carried out.

Therefore, the present invention relates to a process (P4′), which is process (P), (P1), (P1′), (P1″), (P2), (P2′), (P2″), (P2″′), (P3), (P3′) or (P3″), wherein the granulation process is performed for at least 30 minutes (up to several hours) and subsequent drying phase of at least 30 minutes (up to 2 h) at a product temperature of 75° C. to 90° C. is carried out.

Therefore, the present invention relates to a process (P4″), which is process (P), (P1), (P1′), (P1″), (P2), (P2′), (P2″), (P2′″), (P3), (P3′) or (P3″), wherein the granulation process is performed for at least 30 minutes (up to several hours) and subsequent drying phase of at least 30 minutes (up to 2 h) at a product temperature of around 80° C. is carried out.

The following Example illustrates the invention further without limiting it. All percentages and parts, which are given, are related to the weight and the temperatures are given in ° C., when not otherwise stated.

### EXAMPLES

### Example 1

16.5 g β-carotene and 1.65 g D/L-α-tocopherol were suspended into 210 mL isobutyl acetate using a 500 mL three-necked round bottom flask and heated-up to 115° C. for 90 min to prepare the solution. For the matrix phase containing the emulsifier, 120 g OSA-modified food starch were dissolved in 260 mL water at room temperature in 1 L beaker using conventional vertical stirring for 1 h. The solution is added to the matrix phase and emulsified at 50-60° C. for up to 9 min using a rotor stator IKA G45M with high shear stirrer IKA T50 at 10.000 rpm.

After transferring the emulsion into a 2 L three-necked round bottom flask and the addition of 125 ml water, the organic solvent was evaporated at 40° C. under vacuum. Vacuum evaporation and eventual further water addition were continued until reaching a final emulsion with appropriate levels of viscosity and residual moisture for the subsequent drying process.

The emulsion was spray-granulated in two-steps by i) the preparation of seed material applying spray drying with an air inlet temperature of 180° C. and an air outlet temperature of 80° C. in a GEA Niro spray tower, Mobile Minor 2000 Model D1-Spezial and ii) the preparation of granules using a fluid bed processor type WFP-Mini (DMR Prozess-Technologie GmbH). The granulation process via the spraying of emulsion onto the seed particles was performed for 3 h and a subsequent drying phase for 2 h at a product temperature of 80° C. The spray granulation process yielded an increase of the (all-E)-isomeric ratio from 50-52% of the liquid emulsion to 70% of the final granulated product.

### Example 2

33 g β-carotene and 3.3 g D/L-α-tocopherol were suspended into 420 mL isobutyl acetate using a 1 L three-necked round bottom flask and heated-up to 115° C. for 90 min to prepare the solution. For the matrix phase containing the emulsifier, 240 g OSA-modified food starch were dissolved in 520 mL water at room temperature in 2 L beaker using conventional vertical stirring for 1 h. The solution is added to the matrix phase and emulsified at 50-60° C. for up to 18 min using a rotor stator IKA G45M with high shear stirrer IKA T50 at 10.000 rpm.

After transferring the emulsion into a 4 L three-necked round bottom flask and the addition of 250 ml water, the organic solvent was evaporated at 40° C. under vacuum. Vacuum evaporation and eventual further water addition were continued until reaching a final emulsion with appropriate levels of viscosity and residual moisture for the subsequent drying process.

The liquid emulsion was spray-granulated in two-steps by i) the preparation of seed material applying spray drying with an air inlet temperature of 180° C. and an air outlet temperature of 80° C. in a GEA Niro spray tower, Mobile Minor 2000 Model D1-Spezial and ii) the preparation of granules using a fluid bed processor type WFP-Mini (DMR Prozess-Technologie GmbH). The granulation process via the spraying of emulsion onto the seed particles was performed for 3 h and a subsequent drying phase for up to 2 h at a product temperature of 80° C. The spray granulation process yielded an increase of the (all-E)-isomeric ratio from 50-52% of the liquid emulsion to 70% of the final granulated product.

### Example 3

The liquid emulsion was prepared according to workflows as described in the Examples 1 and 2. The liquid emulsion was spray-granulated in two-steps by i) the preparation of seed material applying spray drying with an air inlet temperature of 180° C. and an air outlet temperature of 80° C. in a GEA Niro spray tower, Mobile Minor 2000 Model D1-Spezial and ii) the preparation of granules using a fluid bed processor type WFP-Mini (DMR Prozess-Technologie GmbH). The granulation process via the spraying of emulsion onto the seed particles was performed for 1.5 h and a subsequent drying phase for 1 h at a product temperature of 80° C. The spray granulation process yielded an increase of the (all-E)-isomeric ratio from 52-55% of the liquid emulsion to 63-64% of the final granulated product.

### Example 4

The liquid emulsion was prepared according to workflows as described in the Examples 1 and 2. The liquid emulsion was spray-granulated in two-steps by i) the preparation of seed material applying spray drying with an air inlet temperature of 180° C. and an air outlet temperature of 80° C. in a GEA Niro spray tower, Mobile Minor 2000 Model D1-Spezial and ii) the preparation of granules using a fluid bed processor type WFP-Mini (DMR Prozess-Technologie GmbH). The granulation process via the spraying of emulsion onto the seed particles was performed for 1.5 h and a subsequent drying phase for 2 h at a product temperature of 80° C. The spray granulation process yielded an increase of the (all-E)-isomeric ratio from 52-55% of the liquid emulsion to 64% of the final granulated product.

### Example 5 (Comparative Example)

The liquid emulsion was prepared according to workflows as described in the Examples 1 and 2. However, the liquid emulsion was not spray granulated in this comparative example, but only applied to spray drying with an air inlet temperature of 180° C. and an air outlet temperature of 80° C. in a GEA Niro spray tower, Mobile Minor 2000 Model D1-Spezial.

The spray drying process yielded an increase of the (all-E)-isomeric ratio from 52% of the liquid emulsion to only 53% of the spray dried product.

### Example 6 (Comparative Example)

The liquid emulsion was prepared according to workflows as described in the Examples 1 and 2. However, the liquid emulsion was not spray granulated in this comparative example, but only applied to spray drying with an air inlet temperature of 180° C. and an air outlet temperature of 80° C. in a GEA Niro spray tower, Mobile Minor 2000 Model D1-Spezial.

The spray drying process yielded an increase of the (all-E)-isomeric ratio from 52% of the liquid emulsion to only 54% of the spray dried product.

TABLE 1

| | Ex. 1 | Ex. 2 | Ex. 3 | Ex. 4 | Ex. 5 | Ex. 6 |
|---|---|---|---|---|---|---|
| OSA-modified | 82.5 | 82.5 | 82.5 | 82.5 | 82.5 | 82.5 |
| food starch | | | | | | |
| β-carotene | 11.3 | 11.3 | 11.3 | 11.3 | 11.3 | 11.3 |
| D/L-α-tocopherol | 1.1 | 1.1 | 1.1 | 1.1 | 1.1 | 1.1 |
| Residual Moisture | 5.1 | 5.1 | 5.1 | 5.1 | 5.1 | 5.1 |

1. Process for the production of a solid formulation comprising
(i) b-carotene, and
(ii) at least one hydrocolloid, and
(iii) an antioxidant, and
(iv) optionally at least one additional ingredient,
(v) optionally a coating layer,
characterised in that at least 55 weight-% (wt-%) of the b-carotene (based on the total weight of the b-carotene) is in the all-E form,
wherein the liquid emulsion is spray-granulated in two steps:
i) the preparation of seed material applying spray drying with an air inlet temperature of 160 to 190° C. and an air outlet temperature of 70° C.-100° C. and
ii) the preparation of granules using a fluid bed processor.

2. Process according to claim 1, wherein the granulation process is performed for at least 30 minutes (up to several hours) and subsequent drying phase of at least 30 minutes (up to 2 h) at a product temperature of 70° C.-100° C. is carried out.

3. A solid formulation comprising
(i) b-carotene, and
(ii) at least one hydrocolloid, and
(iii) an antioxidant, and
(iv) optionally at least one additional ingredient,
(v) optionally a coating layer,
characterised in that at least 55 weight-% (wt-%) of the b-carotene (based on the total weight of the b-carotene) is in the all-E form, and
wherein the solid formulation has been obtained by spray-granulation from a liquid emulsion comprising at least the ingredients of said solid formulation (i) through (iii).

4. The solid formulation according to claim 3, wherein the content of the b-carotene in the all-E form is at least 58 wt-%, based on the total weight of the b-carotene.

5. The solid formulation according to claim 3, wherein the content of the b-carotene in the all-E form is at least 60 wt-%, based on the total weight of the b-carotene.

6. The solid formulation according to claim 3, wherein the content of the b-carotene in the solid formulation is 0.5 to 25 wt-%, based on the total weight of the solid formulation.

7. The solid formulation according to claim 3, wherein the content of the b-carotene in the solid formulation is 1 to 25 wt-%, based on the total weight of the solid formulation.

8. The solid formulation according to claim 3, wherein the at least one hydrocolloid is chosen from the group consisting of polysaccharides, gelatin of low bloom, medium bloom or high bloom from fish, pork, or bovine, caseins/caseinates, plant- or animal-based protein, other proteinaceous hydrocolloids, and lignosulfonate.

9. The solid formulation according to claim 3, wherein the at least one hydrocolloid is chosen from the group consisting of xanthan gum, gum acacia, pectin, guar gum, caroub gum, alginates, celluloses, cellulose derivatives, such as starch and starch derivatives.

10. The solid formulation according to claim 3, wherein the hydrocolloid is modified food starch.

11. The solid formulation according to claim 3, wherein the content of the at least one hydrocolloid is 40 to 90 wt-%, based on the total weight of the solid formulation.

12. The solid formulation according to claim 3, wherein the antioxidant is tocopherol and wherein the content of the tocopherol is 0.1 to 5 wt.-%, based on the total weight of the solid formulation.

13. A solid formulation obtained by the process according to any of the claim 1.

14. A food product, a beverage, an animal feed product, a consumer product, or a health product comprising said formulation according to claim 3.
